(19) United States
(12) Patent Application Publication
(10) Pub. No.: US 2002/0004666 A1
(43) Pub. Date: Jan. 10, 2002

(54) Catheter arrangement

(76) Inventors: Michael Schwager, Winterthur (CH); Erwin Berger, Stettfurt (CH)

Correspondence Address: SHINJYU GLOBAL IP COUNSELORS, LLP, 1233 20TH STREET, NW, SUITE 700, WASHINGTON, DC, 20036-2680, US

(21) Appl. No.: 09/860,823

(22) Filed: May 21, 2001

(30) Foreign Application Priority Data
May 22, 2000 (DE) 10025266.4

(51) Int. Cl. A61M 29/00

(52) U.S. Cl. 606/191

(57) ABSTRACT

A catheter arrangement (1) is provided with a guide wire tube (3) for receiving a guide wire and an expandable balloon (7) fastened to the guide wire tube (3). The guide wire tube (3) has a bent tip (2) projecting beyond the distal end (4) of the balloon (7).

### BACKGROUND OF THE INVENTION

1. Field of the Invention

This invention generally relates to a catheter arrangement. More specifically, the present invention relates to a catheter arrangement comprising a guide wire tube for receiving a guide wire and an expandable balloon fastened to the guide wire tube.

2. Background Information

A catheter arrangement is used for implanting stents at places of constriction in body vessels or body cavities so as to expand and stabilize the places of constriction. A conventional catheter arrangement preferably comprises an expandable balloon that is inserted into the body vessels or body cavities.

A controllable guide wire is typically used for guiding the catheter. However, problems arise in the conventional catheter arrangement when the catheter is to be introduced into a vessel branch. The guide wire is pushed forwards as much as possible to ensure a stiff guidance in the area of the branch. However, a deep anchorage of the guide wire in the branch is often not possible for anatomical reasons. Since the stiffness of the guide wire only increases with an increasing distance from the tip of the wire, it can very often not be ruled out that only the soft guide wire tip is available as a guide in the branch.

The catheter to be pushed forwards is much stiffer than the guide wire tip. Thus, the guide wire cannot exert a sufficiently great bending force on the catheter. This results in the guide wire often being pressed out of the vessel branch in the conventional catheter arrangement when the stiffer catheter is pushed forwards along the guide wire towards the vessel branch.

In view of the above, there exists a need for a catheter which overcomes the above mentioned problems in the prior art. This invention addresses this need in the prior art as well as other needs, which will become apparent to those skilled in the art from this disclosure.

### SUMMARY OF THE INVENTION

It is therefore the object of the present invention to provide a catheter arrangement which allows a safe insertion into vessel branches without the risk that the guide wire is pressed out of the vessel branch.

This object is achieved by providing a catheter arrangement including a guide wire tube for receiving a guide wire and an expandable balloon. The guide wire tube comprises a bent tip projecting beyond the distal end of the balloon. This guarantees a reliable insertion into branches of a vessel system because the guide wire which has previously been pushed forwards into the branch can no longer be pressed out of the branch because of the bent tip of the catheter arrangement; rather, the catheter arrangement threads into the branch without any problems.

In accordance with one aspect of the present invention, the guide wire tube of the catheter arrangement has a tip with a radius of curvature of about 7 mm. Furthermore, the length of the bent section of the tip is at least 4 mm, and preferably more than about 5 mm.

These and other objects, features, aspects and advantages of the present invention will become apparent to those skilled in the art from the following detailed description, which, taken in conjunction with the annexed drawings, discloses a preferred embodiment of the present invention.

### BRIEF DESCRIPTION OF THE DRAWINGS

Referring now to the attached drawings which form a part of this original disclosure:

FIG. 1 is a slightly simplified schematic cross sectional view through a catheter arrangement according to one embodiment of the present invention;

FIG. 2 is a strongly simplified schematic cross sectional of the catheter arrangement of FIG. 1, showing a mode of operation of the catheter arrangement of the present invention; and

FIG. 3 is a strongly simplified schematic cross sectional of the catheter arrangement of FIG. 1, showing another mode of operation of the catheter arrangement of the present invention.

### DETAILED DESCRIPTION OF THE PREFERRED EMBODIMENTS

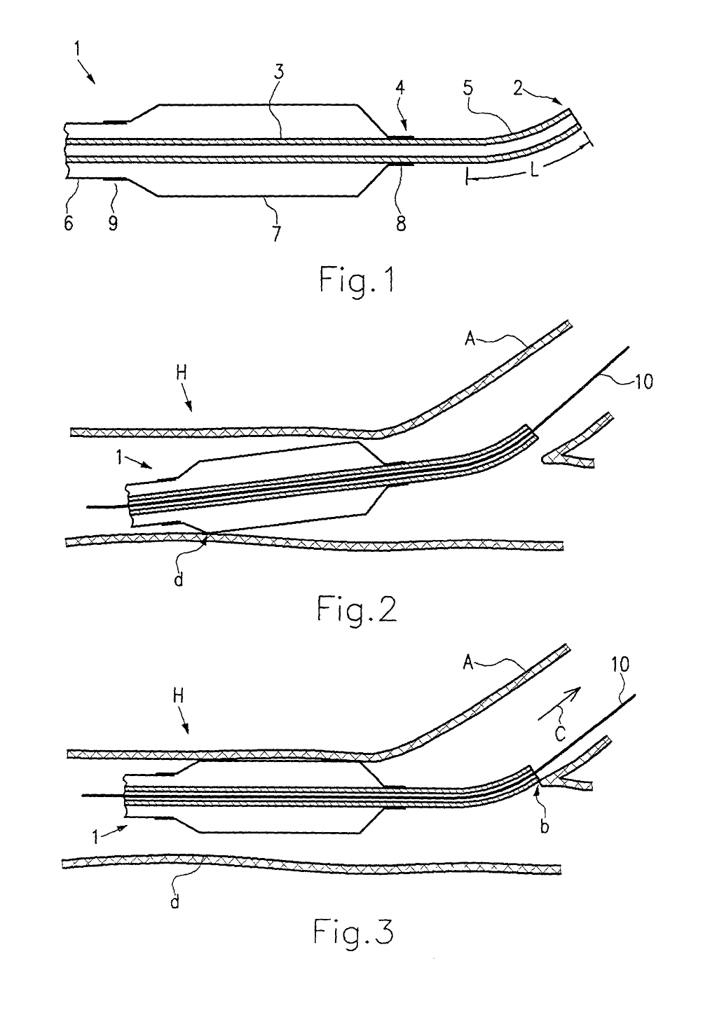

FIG. 1 shows a possible example of a catheter arrangement 1 according to one embodiment of the present invention. The catheter arrangement 1 is used for the implantation of a stent (not shown). The catheter arrangement 1 comprises a guide wire tube 3 in which a guide wire 10 can be slideably arranged. The guide wire 10 and the guide wire tube 3 form a joint guide means.

As shown in FIG. 1, the catheter arrangement 1 further comprises a balloon 7 and a supply tube 6 (only partially shown). The balloon 7 is fastened at its distal end to the guide wire tube 3 via a distal fastening portion 8 and at its proximal end to the tube 6 via a proximal fastening portion 9. “Distal end” means “leading towards the heart” and “proximal end” means “leading away from the heart”. The tube 6 that is connected to the balloon 7 serves to supply a suitable medium such as a saline solution into the hollow interior of the balloon 7 and to expand the balloon at the place of constriction. A stent (not shown) which is mounted around the balloon 7 in a conventional manner can thereby be expanded and can, in turn, stabilize the expansion of the constricted place in the hollow vessel of the body as a vessel support.

Furthermore, the catheter arrangement 1 according to the present invention is provided at its distal end 4 with a bent tip 2 which is an extension of the guide wire tube 3 projecting beyond the distal end 4 of the balloon 7.

Preferably, the bent tip 2 has a radius of curvature of about 7 mm. As a rule, however, it is possible to provide different radii of curvature, depending on the use intended for the catheter arrangement 1 of the present invention.

Depending on the respective application, the length L of the bent section 5 of the bent tip 2 is at least 2 mm and more, but in a particularly preferred embodiment is at least more than about 5 mm.

FIGS. 2 and 3 illustrate the modes of operation of the catheter arrangement 1 according to the present invention. FIGS. 2 and 3 are strongly simplified schematic illustrations showing a hollow vessel H into the branch A of which the catheter arrangement 1 is to be placed. To this end a guide wire 10 is first pushed forwards into the branch A and the catheter arrangement 1 is subsequently placed via the guide wire 10 into the branch A. FIGS. 2 and 3 show that the bent tip 2 ensures the advance movement into the branch A because the much stiffer catheter arrangement 1 can no longer press the guide wire 10 out of the branch A on account of the bent tip 2 in cases where the guide wire 10 cannot deeply be anchored in said branch A, e.g. for anatomical reasons. The bent tip 2 offers the advantage that the catheter arrangement 1 can be introduced into the branch A without a force being exerted by the guide wire 10. Typically, according to the illustration shown in FIG. 3, the bent tip 2 impinges on point b so that during the further advance movement the tip 2 slides further forwards on the inner wall of the vessel in direction C.

When the tip 2 does not slide right away in direction C for reasons of friction, the catheter arrangement 1 is bent around the point of contact b and contacts the inner wall of the vessel at point d, which becomes apparent from FIG. 2. This, in turn, creates a reactive force on the catheter arrangement 1 towards branch A, which permits the advance movement in direction C without the risk that the guide wire 10 is pressed out of the hollow vessel H.

The terms of degree such as “substantially”, “about” and “approximately” as used herein mean a reasonable amount of deviation of the modified term such that the end result is not significantly changed. These terms should be construed as including a deviation of at least ±5% of the modified term if this deviation would not negate the meaning of the word it modifies.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing description of the embodiments according to the present invention are provided for illustration only, and not for the purpose of limiting the invention as defined by the appended claims and their equivalents.

What is claimed is:

1. A catheter arrangement comprising
a guide wire tube for receiving a guide wire, said guide wire tube having a proximal end and a distal end; and
an expandable balloon coupled to said guide wire tube, said distal end of said guide wire tube having a bent tip projecting beyond a distal end of said balloon.

2. The catheter arrangement according to claim 1, wherein said bent tip has a radius of curvature of about 7 mm.

3. The catheter arrangement according to claim 2, wherein said bent tip has a length in an axial direction of at least 4 mm.

4. The catheter arrangement according to claim 2, wherein said bent tip has a length in an axial direction of more than 5 mm.

5. The catheter arrangement according to claim 1, wherein said bent tip has a length in an axial direction of at least 4 mm.

6. The catheter arrangement according to claim 5, wherein said bent tip has a length in an axial direction of more than 5 mm.
